(19) United States
(12) Patent Application Publication
(10) Pub. No.: US 2017/0135942 A1
(43) Pub. Date: May 18, 2017

(54) Compositions for Keratin Fibers

(71) Applicant: Avon Products, Inc., Suffern, NY (US)

(72) Inventors: Candice DeLeo Novack, Monroe, NY (US); Kelly Hales, Nanuet, NY (US)

(21) Appl. No.: 14/943,605

(22) Filed: Nov. 17, 2015

(51) Int. Cl. A61K 8/88; A61K 8/87; A61K 8/81; A61K 8/37; A61Q 1/10; A61K 8/92

(52) U.S. Cl. CPC A61K 8/88; A61Q 1/10; A61K 8/927; A61K 8/8111; A61K 8/375; A61K 8/87; A61K 2800/43; A61K 2800/524; A61K 2800/48; A61K 2800/49; A61K 2800/592

(57) ABSTRACT

The present invention relates generally to compositions and methods of using them to treat keratin fibers. More particularly, the compositions according to the invention, such as mascaras and other cosmetics, comprise polyamide gelling agents and soft waxes, and are useful for imparting the appearance of enhanced length and volume to the treated keratin fibers.

### FIELD OF INVENTION

The present invention relates generally to compositions and methods of using them to treat keratin fibers. More particularly, the compositions according to the invention, such as mascaras and other cosmetics, comprise polyamide gelling agents and soft waxes, and are useful for imparting the appearance of enhanced length and volume to the treated keratin fibers.

### BACKGROUND OF THE INVENTION

Mascaras are generally used by consumers to accentuate the eyelashes by imparting color and/or aesthetic effects to the eyelashes. In particular, consumers use mascaras to darken, thicken, lengthen, and curl their lashes. With presently marketed mascaras, thickening of the eyelashes is typically achieved by incorporating in such products a high level of hard waxes and film formers to impart a thick film on the lashes. These formulations tend to clump on the lashes, causing them to stick together, and over time become brittle and subject to flaking. Mascaras that purport to lengthen the lashes often rely on fiber-containing formulas, whereby the fibers attach to the end of the lash and orient in the direction of the lash in order to achieve a lengthening effect. Lacking in the art are mascaras and other cosmetics for treating keratin fibers, which both lengthen and volumize the lashes while remaining flexible and resistant to flaking, and which do not rely on the use of fibers in the composition. It is therefore an object of the invention to provide compositions and methods for treating keratin fibers which achieve both volumizing and lengthening effects, ideally without flaking and clumping of the lashes.

### SUMMARY OF THE INVENTION

Compositions and methods for treating keratin fibers (e.g., eyelashes) are provided. The compositions are characterized by an ability to both lengthen and volumize the appearance of keratin fibers such as eyelashes. The compositions have a diminished propensity for clumping and flaking.

In one aspect of the invention, compositions (e.g., pigmented compositions, such as mascaras) are provided, typically comprising an oil, a polyamide gellant for forming a gel with the oil, a soft wax, an associative rheological modifier, and a polyurethane film former. The oil (e.g., a hydrocarbon, ester oil, or fatty alcohol, including saturated or partially unsaturated C₆₋₃₀ or C₁₂₋₂₆ fatty alcohols and branched fatty alcohols such as octyldodoecanol) is typically one which is capable of forming a gel with the polyamide gellant. In some implementations, the compositions may comprise polydecene and/or gylceryl rosinate. The oil may be present in an amount between about 0.1% and about 20% (e.g., between about 0.5-20%, between about 1%-15%, or between about 5%-10%) by weight of the composition. The gellant may be any polyamide gellant capable of forming a gel with an oil, for example, ester-terminated polyamides (ETPA), ester-terminated poly(ester-amide) polymeric gellants (ETPEA) (e.g., Bis-Stearyl Ethylenediamine/Neopentyl Glycol/Stearyl Hydrogenated Dimer Dilinoleate copolymer (INCI)), tertiary amide terminated polyamides (ATPA), polyalkyleneoxy terminated polyamides (PAOPA), and polyether polyamides (PEPA). The polyamide gellant may be present in an amount between about 0.01% and about 10% (e.g., between about 0.01%-5%, or between about 0.05%-3%, between about 0.1%-2%, or between about 0.5%-1%) of the composition. The composition typically includes a soft wax, which may, for example, have a melting point below 75° C., or below 72.5° C., or below 70° C. In certain implementations, the soft wax will include one or more of paraffin wax, ozokerite, lanolin wax, candelilla wax, silicone wax, beeswax and modified beeswax derivatives such as bleached beeswax, sorbitol beeswax, silconyl beeswax, and PEG-modified beeswax (e.g., PEG-8 beeswax and PEG-3 beeswax). The soft wax may be present individually or in the aggregate, in an amount between about 1% and about 40% (e.g., between about 5%-30%, between about 7.5%-25%, or between about 10%-20%) by weight of the composition. In one embodiment, the composition also comprises a hard wax in combination with the low melting point wax. A hard wax may, for example, have a melting point above about 75° C. (or above 80° C., or above 85° C., etc.). In some implementations, a hard wax, if included, is present in an amount, individually or in the aggregate, less than about 5%, or less than about 3%, or less than about 2%, or less than about 1%, or less than about 0.5% by weight. In another embodiment, the composition may be substantially free of (e.g., comprise less than 1%, or less than 0.1%, or less than 0.05%) or free of a hard wax. The composition may also comprise one or more associative rheological modifiers/thickeners, such as ethoxylated polyurethanes, cross-linked acrylate copolymers, and acrylic acid homopolymers, as well as any suitable nonionic, cationic, anionic or amphoteric thickeners. In one implementation, the associative thickener comprises an alkali-swellable acrylic emulsion polymer, such as Acrylates Copolymer (INCI). The associative rheological modifier may be present, individually or collectively, in an amount between about 0.1% and about 20% (e.g., between about 0.5%-15%, or between about 1%-10%, or between about 2%-8%) by weight of the composition. The composition may also comprise a film former, such as polyurethane film formers (i.e., those that are formed by reacting a di- or polyisocyanate with a diol and/or polyol), including for example, aqueous polyurethane dispersions. In one implementation, the film former may comprise a copolymer of adipic acid, dicyclohexylmethane diisocyanate, ethylenediamine, Hexandiol, Neopentyl Glycol and sodium N-(2-aminoethyl)-3-aminoethanesulfonate monomers (INCI: Polyurethane-35). The film formers may be present, individually or collectively, in an amount between about 0.1% and about 50% (e.g., between about 1%-50%, or between about 2%-45%, or between about 5%-35%, or between about 6% to about 30%, or between about 8%-25%, or about 10%-20%) of the composition. The composition may also comprise one or more particulates (e.g., any suitable particulate fillers, clays, gums, fibers, polymers, powders, lakes, or pigments), which may be present individually or in the aggregate in an amount between about 0.01% and about 30% (e.g., between about 0.1%-25%, between about 1%-20%, between about 3%-15%, or between about 5%-10%) by weight of the composition. In some implementations, the compositions will comprise one or both of carbon black and iron oxide, each of which may be present, individually or in the aggregate with all other particulates, in the foregoing amounts. In one embodiment, the composition may further comprise, in addition to the polyamide gellant, one or more glutamide-based oil-phase gellants, such as dibutyl laurolyl glutamide and/or dibutyl ethylhexanoyl glutamide, in an amount individually or collectively between about 0.001% and about 10% (e.g., between about 0.01%-5%, or between about 0.05%-1%, or between about 0.1%-0.5%) by weight of the composition. The compositions may comprise an aqueous phase, and may be in the form of emulsions (e.g., oil-in-water, water-in-oil, etc.), or they may be substantially anhydrous (e.g., <2%, for example, less than 1%, or less than 0.5%, or less than 0.25% water), or they may be anhydrous.

In another aspect of the invention, methods of using the compositions to impart length and volume to keratin fibers (e.g., eyelashes) are provided. The methods generally comprise applying a composition of the invention (e.g., a pigmented cosmetic such as a mascara) to keratin fibers to cover at least a substantial portion, or at least a major portion thereof and drawing out a portion of the composition beyond the tip of the fiber (e.g., 0.1 mm-1 cm or more). Upon evaporation of volatiles (e.g., partial or complete evaporation of solvents and other volatiles), the composition forms a dry film on the surfaces of the keratin fibers that imparts both increased length and volume to the keratin fibers, while allowing them to remain flexible and resistant to flaking and breakage. In some implementations, the compositions may be characterized as having high extensional viscosity, and exhibit a first normal stress difference of at least 35 Pa, or at least 40 Pa, or at least 45 Pa, or at least 50 Pa, or at least 55 Pa, or at least 60 Pa. In some implementations, the dried films are characterized as being highly flexible, or substantially resistant to loss of the dried film following moving and/or stretching of the treated lashes.

These and other aspects of the present invention will become apparent to those skilled in the art according to the present description, including the figures and appended claims.

### DETAILED DESCRIPTION

As used herein, the term “consisting essentially of” is intended to limit the invention to the specified materials or steps and those materials or steps that do not materially affect the basic and novel characteristics of the claimed invention, for example, the extensional viscosity of the compositions as understood from a reading of this specification.

The terms “a” and “an,” as used herein and in the appended claims, mean “one or more” unless otherwise indicated. It should be noted that unless otherwise indicated, percent (%) is % by weight, based on the total weight of the composition (including any solvents or vehicle). It will be understood that the weight % of all components, in the aggregate, will not exceed 100%. Unless otherwise indicated, each component may be included in the compositions in amounts ranging from about 0.0001% by weight to about 20% by weight (e.g., 0.001-10% by weight). Any solvents or other vehicle used in the compositions of the invention are topically acceptable, by which is meant non-toxic and substantially non-irritating to human integuments.

The compositions of the invention are intended for application to any human integument, including skin, nails, lips, hair, lashes, etc. In some embodiments, the compositions are intended for both lengthening and volumizing keratin fibers, (e.g., eyelashes, hair of the scalp, etc.), most notably eyelashes. The compositions of the invention, such as pigmented mascaras, are generally characterized by a unique ability to impart both length and volume to treated keratin fibers while remaining flexible, and without substantial loss of the film caused by brittleness, breakage, and/or flaking.

The compositions of the invention typically comprise an oil, a polyamide gellant capable of forming a gel with the oil, a soft wax, an associative rheological modifier, and a polyurethane film former.

The polyamide gellant may be any polyamide gellant that is capable of gelling or structuring an oil phase. The polyamide gellant may comprise, for example, ester-terminated polyamides (ETPA), ester-terminated poly(ester-amide) polymeric gellants (ETPEA), tertiary amide terminated polyamides (ATPA), polyalkyleneoxy terminated polyamides (PAOPA), and/or polyether polyamides (PEPA).

In some embodiments, the polyamide gellant may comprise an ETPEA gellant such as those disclosed in U.S. Pat. Nos. 6,875,245; 7,253,249; and 7,329,719, which are hereby incorporated by reference in their entirety. Such ETPEA gellants may be prepared, for example, by reacting components comprising dibasic acid, diamine, polyol and monoalcohol, wherein at least 50 equivalent percent of the dibasic acid comprises polymerized fatty acid; and at least 50 equivalent percent of the diamine comprises ethylene diamine. In one embodiment, the polyamide gellant comprises OleoCraft LP-20 (INCI: Poylamide-8; Bis-Stearyl Ethylenediamine/Neopentyl Glycol/Stearyl Hydrogenated Dimer Dilinoleate Copolymer) (Croda Int'l).

In other embodiments, the polyamide gellant may comprise a PAOPA gellant such as those disclosed in U.S. Pat. Nos. 6,956,099; 8,114,387; and 8,114,424 which are hereby incorporated by reference in their entirety. Such PAOPA gellants may include copolymers having linked internal polyether blocks and internal polyamide blocks. For example, the polyamide gellant may comprise OleoCraft HP-31 (INCI: Poylamide-3) (Croda Int'l); Oleocraft MP-30 (INCI: Polyamide 3) (Croda Int'l); and OleoCraft MP-32 (INCI: Polyamide 3) (Croda Int'l). Other suitable PAOPA gellants include those disclosed in U.S. Pat. No. 6,399,713, which is hereby incorporated by reference in its entirety. Such PAOPA gellants may include those comprising a block copolymer of the formula hydrocarbon-polyether-polyamide-polyether-hydrocarbon. For example, the PAOPA gellant may comprise OleoCraft HP-33 (INCI: Polyamide 4) (Croda Int'l).

A suitable ETPA gellant may comprise, for example, Uniclear 100VG (INCI: Ethylenediamine/Stearyl Dimer Dilinoleate Copolymer) (Croda Int′ 1).

A suitable ATPA gellant may comprise, for example, Sylvaclear A200V (INCI: Ethylenediamine/Hydrogenated Dimer Dilinoleate Copolymer Bis-Di-C14-18 Alkyl Amide) (Croda Int′ 1). A suitable PEPA gellant may comprise, for example, Sylvaclear PE400V (INCI: Polyamide 6) (Croda Int'l). A composition “consisting essentially of” a particular polyamide gellant, such as an ETPEA gellant, is intended to mean that the presence of additional polyamide gellants, in amounts which would measurably affect the volume, length, flexibility, and/or extensional viscosity of the composition are excluded.

The polyamide gellant will typically be present in an amount sufficient to impart structure to the composition (i.e., to have a measurable impact on viscosity, for example, ±5% or more). The polyamide gellant may be present in an amount sufficient to provide a viscosity to the composition of at least 1,000 cps, at least 2,500 cps, at least 5,000 cps, at least 10,000 cps, at least 25,000 cps, at least 50,000 cps, at least 100,000 cps, or at least 200,000 cps at 25° C. In some embodiments, the polyamide gellant will be present in an amount from about 0.001 to about 10%, from about 0.01 to about 5%, from about 0.1 to about 5%, or from 0.1% to about 1%, by weight of the composition. In some specific embodiments, the polyamide gellant is present in an amount of about 0.1%, about 0.5%, about 1%, about 0.4%, about 2%, about 3%, about 4%, about 5%, about 6%, or about 7%, or about 8%, or about 9%, or about 10% by weight of the composition.

The composition may also comprise additional oil phase and/or aqueous phase gellants. In one embodiment, the composition will comprise one or more glutamide-based oil-phase gellants, such as dibutyl lauryl glutamide and/or dibutyl ethylhexanoyl glutamide and combinations thereof. In some embodiments the composition may comprise dibutyl lauroyl glutamide. In other embodiments the composition may comprise dibutyl ethylhexanoyl glutamide. Dibutyl lauroyl glutamide and dibutyl ethylhexanoyl glutamide (are available from Ajinomoto Co., Inc. as GP-1 and EB-21, respectively.

Additional suitable gellants and solvents therefore are described in U.S. Pat. No. 7,989,002, the entire contents of which are hereby incorporated by reference. Also suitable are the gellants and solvents therefore as described in U.S. Pat. No. 7,682,621, the entire contents of which are hereby incorporated by reference. Additional oil-phase and aqueous-phase gellants may be included, individually or in the aggregate in an amount from about 0.001% to about 20%, or from about 0.01% to about 10% by weight of the composition.

The composition typically includes one or more soft waxes. As used herein, a “soft wax” is one having a melting point below 75° C., though more typically it has a melting point below 72.5° C. or below 70° C. The melting values provided herein refer to the mid-point of the melting range. In some embodiments, the soft waxes will have an onset of melting temperature below 75° C. Each of the melting values disclosed herein may refer to the midpoint of the melting range, onset of the melting range, or point at which the wax is completely melted.

Table 1 provides representative suitable soft waxes arranged by approximate melting point or melting range.

TABLE 1

| Wax | Melting Point (° C.) |
|---|---|
| esparto wax | 73 |
| ozokerite wax | 72 |
| jojoba wax | 70 |
| candelilla wax | 68-73 |
| ceresin wax | 67-71 |
| beeswax | 62-64 |
| castor wax | 60 |
| sugarcane wax | 60 |
| stearyl alcohol | 59 |
| hard tallow | 57-60 |
| cetyl alcohol | 56 |
| petrolatum | 54 |
| glyceryl monostearate | 54-56 |
| Japan wax | 53 |
| silicone waxes | 53-75 |
| paraffin wax | 50-60 |
| lanolin alcohol | 45-60 |
| bayberry wax | 45 |
| cetyl palmitate | 43-53 |
| lanolin | 38-42 |
| illipe butter | 34-38 |
| cocoa butter | 31-35 |

In one embodiment, the composition comprises one or more hard waxes in combination with the one or more soft waxes. A hard wax may, for example, have a melting point above 75° C., or above 80° C., or above 85° C., or above 90° C., or above 95° C.

Table 2 provides representative suitable hard waxes arranged by approximate melting point or melting range.

TABLE 2

| Wax | Melting Point (° C.) |
|---|---|
| acrawax | 140 |
| microcrystalline petroleum wax | 99 |
| linear polyethylene wax | 95 |
| stearone | 89 |
| castor wax | 86 |
| montan wax | 82-95 |
| lignite wax | 82-95 |
| ouricouri wax | 81-84 |
| carnauba wax | 78-85 |
| rice bran wax | 77-86 |
| shellac wax | 74-78 |

It will be understood that the melting points and ranges provided in Table 1 and Table 2 are merely representative of typical values for each wax, and wide variation in the melting point or melting point range may be observed from sample to sample depending on the source and purity of the wax. It is within the skill in the art to determine the melting point or melting point range of any given wax sample. Melting points may be determined, for example, by DSC, or by drop melting point according to ASTM D127, incorporated by reference herein, and/or ring-and-ball softening point according to ASTM D36, incorporated by reference herein. In the event of a discrepancy between the techniques, melting point will be determined by DSC.

Because some waxes may have broad melting ranges, they may be considered as suitable soft waxes or suitable hard waxes, depending on the particular composition and the desired viscosity. For example, ozokerite may be considered a soft wax in the practice of the invention, but because it is toward the high end of the melting point range for soft waxes, in some embodiments, the composition will be substantially free of ozokerite, by which is meant it is present in amounts insufficient to measurably contribute to the viscosity of the composition (e.g., comprises less than 1% or less than 0.1% or less than 0.01% by weight). Similarly, although shellac wax may be considered a hard wax based on Table 2, because it has a broad melting point range, it may also be useful as a soft wax in some embodiments.

The waxes may be natural, mineral and/or synthetic waxes. Natural waxes are those of animal origin, including without limitation beeswax, spermaceti, lanolin, and shellac wax, and those of vegetable origin, including without limitation carnauba, candelilla, bayberry, and sugarcane wax.

Mineral waxes contemplated to be useful include, without limitation ozokerite, ceresin, montan, paraffin, microcrystalline, petroleum, and petrolatum waxes.

Synthetic waxes include, for example, polyethylene glycols such as PEG-18, PEG-20, PEG-32, PEG-75, PEG-90, PEG-100, and PEG-180 which are sold under the tradename Carbowax® (The Dow Chemical Company). Mention may be made of CARBOWAX 1000 which has a molecular weight range of 950 to 1,050 and a melting point of about 38° C., CARBOWAX 1450 which has a molecular weight range of about 1,305 to 1,595 and a melting point of about 56° C., CARBOWAX 3350 which has a molecular weight range of 3,015 to 3,685 and a melting point of about 56° C., and CARBOWAX 8000 which has a molecular weight range of 7,000 to 9,000 and a melting point of about 61° C.

Synthetic waxes also include Fischer Tropsch (FT) waxes and polyolefin waxes, such as ethylene homopolymers, ethylene-propylene copolymers, and ethylene-hexene copolymers. Representative ethylene homopolymer waxes are commercially available under the tradename POLYWAX ° Polyethylene (Baker Hughes Incorporated) with melting points ranging from 80° C. to 132° C. Commercially available ethylene-α-olefin copolymer waxes include those sold under the tradename PETROLITE®. Copolymers (Baker Hughes Incorporated) with melting points ranging from 95° C. to 115° C.

In some embodiments, the composition may comprise one or more waxes selected from the group consisting of paraffin wax, ozokerite, silicone wax, beeswax and beeswax derivatives such as bleached beeswax, sorbitol beeswax, siliconyl beeswax, and PEG-modified beeswax, PEG-8 beeswax and PG-3 beeswax, lanolin, candelilla wax, and silicone waxes, such as SILWAX CRM2, SILWAX 5022, SILWAX L118, SILWAX D221M, and SILWAX Di-5026. These waxes may be present, individually or in the aggregate, in an amount from about 0.1%-40% by weight, or 0.5%-25% by weight, or 1%-15% by weight of the composition.

In one embodiment, the composition is free of, or substantially free of jojoba wax. In another embodiment, the composition is free of, or substantially free of ozokerite wax. In another embodiment, the composition is free of, or substantially free of candelila wax. In another embodiment, the composition is free of, or substantially free of ceresin wax. In another embodiment, the composition is free of, or substantially free of carnauba wax. As used herein, “substantially free” means that the wax is present in amounts insufficient to measurably contribute to the viscosity of the composition (e.g., comprises less than 1%, or less than 0.1% or less than 0.01% by weight).

The soft waxes, either individually or in the aggregate, may be present in the compositions in an amount between about 0.01% to about 40%, about 0.1% to about 35%, about 1% to about 30%, about 2.5% to about 25%, or about 5% to about 20% by weight of the composition. In some embodiments, the soft wax component may comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of the composition.

In some embodiments, the composition optionally comprises one or more hard waxes. If present, hard waxes will be present individually or in the aggregate, in an amount between about 0.01% and about 5%, between about 0.1% and about 3%, between about 0.5% and about 2.5%, or between about 1% and about 2% by weight of the composition. In another embodiment, the composition may be “substantially free” of a hard wax, by which is meant less than 1% (w/w). In other embodiments, the compositions are free of hard waxes. As used herein, hard waxes have a melting point above 75° C.

The soft and/or hard wax component may include one or more low opacity waxes (e.g., a ΔL* less than 8 as determined by the procedure set forth below. The hard and soft waxes collectively may have a ΔL* value of less than 8 and/or each individual wax may have a ΔL* value of less than 8. In other embodiments the ΔL* value of the waxes, individually is 10 or less, 9 or less, 8 or less, 6 or less, 5 or less, 4 or less, 3 or less, 2 or less, or 1 or less. In certain embodiments the wax component may comprise one or more individual waxes having a ΔL* less than 8, in combination with one or more waxes individually having a ΔL* of 8 or greater, as long as in the aggregate, the combination of waxes (i.e., the wax component) exhibits a ΔL* less than 8. In one embodiment, the wax component does not comprise an individual wax having a ΔL* of 8 or greater. In other embodiments the wax component does not comprise more than 15%, more than 10%, or more than 5%, or more than 1% of a wax having a ΔL* value of 8 or greater, by weight of the wax component.

ΔL* is measured by measuring L* values on a drawdown film on a black Leneta card using a hand-held spectrophotometer (e.g., a Konica Minolta CM-2600d spectrophotometer). The drawdown film is obtained by applying 3 mL of the sample to obtain a test film on the Leneta card that is about 75 microns in thickness and allowed to dry for 2 hours. The Leneta card itself is the standard for the color black in the tristimulus color measurement method, and by definition has an L value of zero. The ΔL* of the entire composition may be measured using the same protocol.

Suitable low opacity waxes include, but are not limited to, carnauba wax, beeswax, bleached beeswax, ozokerite, kahlwax 7307, and silicone waxes (e.g., SILWAX CRM2, SILWAX 5022, SILWAX L118, SILWAX D221M, SILWAX Di-5026), POE (20M) sorbitol beeswax, PEG-8 beeswax, and other modified beeswax derivatives, variants and combinations thereof.

If included, the low opacity wax may be present individually or in the aggregate, in an amount between about 0.01% and about 25%, between about 1% and about 20%, or between about 5% and about 10% by weight of the composition.

The composition typically comprises one or more oils that is capable of forming a gel with the polymeric gellant and/or with an optional additional oil-phase gellant. Suitable oils include, without limitation, vegetable oils; esters including emollient esters, such as octyl palmitate, isopropyl myristate and isopropyl palmitate; ethers such as dicapryl ether; fatty alcohols such as cetyl alcohol, and branched alcohols like octyldodecanol, stearyl alcohol and behenyl alcohol; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; and the like. Suitable hydrophobic hydrocarbon oils may be saturated or unsaturated, have an aliphatic character and be straight or branched chained or contain alicyclic or aromatic rings. The oil may be composed of a singular oil or mixtures of different oils.

Exemplary hydrocarbon oils may comprise straight or branched chain paraffinic hydrocarbons having from 5 to 80 carbon atoms, typically from 8 to 40 carbon atoms, and more typically from 10 to 16 carbon atoms, including but not limited to, pentane, hexane, heptane, octane, nonane, decane, undecane, dodecane, tetradecane, tridecane, and the like. Some useful hydrocarbon oils are highly branched aliphatic hydrocarbons, including C₈₋₉ isoparaffins, C₉₋₁₁ isoparaffins, C₁₂ isoparaffin, C₂₀₋₄₀ isoparaffins and the like. Special mention may be made of the isoparaffins having the INCI names isohexadecane, isoeicosane, and isododecane (IDD).

Paraffinic hydrocarbons are available from Exxon under the ISOPARS trademark, and from the Permethyl Corporation. In addition, C₈₋₂₀ paraffinic hydrocarbons such as C₁₂ isoparaffin (isododecane) manufactured by the Permethyl Corporation having the tradename PERMETHYL 99A™ are also contemplated to be suitable. Various commercially available C₁₆ isoparaffins, such as isohexadecane (having the tradename PERMETHYL®) are also suitable. Examples of volatile hydrocarbons include polydecanes such as isododecane and isodecane, including for example, PERMETHYL-99A (Presperse Inc.) and the C₇-C₈ through C₁₂-C₁₅ isoparaffins such as the Isopar Series available from Exxon Chemicals.

Also suitable as hydrocarbon oils are poly-alpha-olefins, typically having greater than 20 carbon atoms, including (optionally hydrogenated) C₂₄₋₂₈ olefins, C₃₀₋₄₅ olefins, polyisobutene, hydrogenated polyisobutene, hydrogenated polydecene, polybutene, hydrogenated polycyclopentane, mineral oil, pentahydrosqualene, squalene, squalane, and the like. The hydrocarbon oil may also comprise higher fatty alcohols, such as oleyl alcohol, octyldodecanol, and the like.

Suitable oils may also comprise one or more volatile and/or non-volatile silicone oils. Volatile silicones include cyclic and linear volatile dimethylsiloxane silicones. In one embodiment, the volatile silicones may include cyclodimethicones, including tetramer (D₄), pentamer (D₅), and hexamer (D₆) cyclomethicones, or mixtures thereof. Particular mention may be made of the volatile cyclomethicone-hexamethyl cyclotrisiloxane, octamethyl-cyclotetrasiloxane, and decamethyl-cyclopentasiloxane. Suitable dimethicones are available from Dow Corning under the name DOW CORNING 200® Fluid and have viscosities ranging from 0.65 to 600,000 centistokes or higher. Suitable non-polar, volatile liquid silicone oils are disclosed in U.S. Pat. No. 4,781,917, herein incorporated by reference in its entirety. Additional volatile silicones materials are described in Todd et al., “Volatile Silicone Fluids for Cosmetics,” Cosmetics and Toiletries, 91:27-32 (1976), herein incorporated by reference in its entirety. Linear volatile silicones generally have a viscosity of less than about 5 centistokes at 25° C., whereas the cyclic silicones have viscosities of less than about 10 centistokes at 25° C. Examples of volatile silicones of varying viscosities include DOW CORNING 200, DOW CORNING 244, DOW CORNING 245, DOW CORNING 344, and DOW CORNING 345, (Dow Corning Corp.); SF-1204 and SF-1202 SILICONE FLUIDS (G.E. Silicones), GE 7207 and 7158 (General Electric Co.); and SWS-03314 (SWS Silicones Corp.). Linear, volatile silicones include low molecular weight polydimethylsiloxane compounds such as hexamethyldisiloxane, octamethyltrisiloxane, decamethyltetrasiloxane, and dodecamethylpentasiloxane, to name a few.

Non-volatile silicone oils will typically comprise polyalkylsiloxanes, polyarylsiloxanes, polyalkylarylsiloxanes, or mixtures thereof. Polydimethylsiloxanes are non-volatile silicone oils. The non-volatile silicone oils will typically have a viscosity from about 10 to about 60,000 centistokes at 25° C., in one embodiment between about 10 and about 10,000 centistokes, and in one embodiment still between about 10 and about 500 centistokes; and a boiling point greater than 250° C. at atmospheric pressure. Non-limiting examples include dimethyl polysiloxane (dimethicone), phenyl trimethicone, and diphenyldimethicone. The volatile and non-volatile silicone oils may optionally be substituted with various functional groups such as alkyl, aryl, amine groups, vinyl, hydroxyl, haloalkyl groups, alkylaryl groups, and acrylate groups, to name a few.

In one embodiment, the silicone oil may be a fluorinated silicone, such as a perfluorinated silicone (i.e., fluorosilicones). Fluorosilicones are advantageously both hydrophobic and oleophobic and thus contribute to a desirable slip and feel of the product. Fluorosilicones can be gelled with behenyl behenate if desired. One suitable fluorosilicone is a fluorinated organofunctional silicone fluid having the INCI name Perfluorononyl Dimethicone. Perfluorononyl Dimethicone is commercially available from Phoenix Chemical under the trade name PECOSIL®.

Additional suitable oils may include, for example, isostearyl neopentanoate, isostearyl stearate, castor oil, lauryl lactate, isopropyl palmitate, glyceryl triacethyl hydroxystearate, diisopropyl adipate, octyl isononanoate, neopentyl glycol dioctanoate, neopentyl glycol dicaprate, isodecyl oleate, and myristyl myristate.

The compositions may comprise one or more ester oils. The esters may be, for example, mono-esters, di-esters, or tri-esters. Ideally, the additional esters, if present, also provide emolliency to the composition.

Other suitable additional ester oils that may be used in the compositions of the invention include fatty acid esters, and in particular, those esters commonly used as emollients in cosmetic formulations. Such esters will typically be the esterification product of an acid of the form R₄(COOH)₁₋₂ with an alcohol of the form R₅(OH)₁₋₃ where R₄ and R₅ are each independently linear, branched, or cyclic hydrocarbon groups, optionally containing unsaturated bonds (e.g., from 1-6 or 1-3 or 1), and having from 1 to 30 (e.g., 6-30 or 8-30, or 12-30, or 16-30) carbon atoms, optionally substituted with one or more functionalities including hydroxyl, oxa, oxo, and the like. Preferably, at least one of R₄ and R₅ comprises at least 8, or at least 10, or at least 12, or at least 16 or at least 18 carbon atoms, such that the ester oil comprises at least one fatty chain. The esters defined above will include, without limitation, the esters of mono-acids with mono-alcohols, mono-acids with diols and triols, di-acids with mono-alcohols, and tri-acids with mono-alcohols.

Suitable fatty acid esters include, without limitation, butyl isostearate, butyl oleate, butyl octyl oleate, cetyl palmitate, ceyl octanoate, cetyl laurate, cetyl lactate, cetyl isononanoate, cetyl stearate, diisostearyl fumarate, diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisopropyl dimerate, triisostearyl trilinoleate, octodecyl stearoyl stearate, hexyl laurate, hexadecyl isostearate, hexydecyl laurate, hexyldecyl octanoate, hexyldecyl oleate, hexyldecyl palmitate, hexyldecyl stearate, isononyl isononanaote, isostearyl isononate, isohexyl neopentanoate, isohexadecyl stearate, isopropyl isostearate, n-propyl myristate, isopropyl myristate, n-propyl palmitate, isopropyl palmitate, hexacosanyl palmitate, lauryl lactate, octacosanyl palmitate, propylene glycol monolaurate, triacontanyl palmitate, dotriacontanyl palmitate, tetratriacontanyl palmitate, hexacosanyl stearate, octacosanyl stearate, triacontanyl stearate, dotriacontanyl stearate, stearyl lactate, stearyl octanoate, stearyl heptanoate, stearyl stearate, tetratriacontanyl stearate, triarachidin, tributyl citrate, triisostearyl citrate, tri-C₁₂₋₁₃-alkyl citrate, tricaprylin, tricaprylyl citrate, tridecyl behenate, trioctyldodecyl citrate, tridecyl cocoate, tridecyl isononanoate, glyceryl monoricinoleate, 2-octyldecyl palmitate, 2-octyldodecyl myristate or lactate, di(2-ethylhexyl)succinate, tocopheryl acetate, and the like.

Other suitable esters include those wherein R₅ comprises a polyglycol of the form H—(O—CHR*—CHR*)ₙ— wherein R* is independently selected from hydrogen or straight chain C₁₋₁₂ alkyl, including methyl and ethyl, as exemplified by polyethylene glycol monolaurate.

Salicylates and benzoates are also contemplated to be useful esters in the compositions of the invention. Suitable salicylates and benzoates include esters of salicylic acid or benzoic acid with an alcohol of the form R₆OH where R₆ is a linear, branched, or cyclic hydrocarbon group, optionally containing unsaturated bonds (e.g., one, two, or three unsaturated bonds), and having from 1 to 30 carbon atoms, typically from 6 to 22 carbon atoms, and more typically from 12 to 15 carbon atoms. Suitable salicylates include, for example, octyl salicylate and hexyldodecyl salicylate, and benzoate esters including C₁₂₋₁₅ alkyl benzoate, isostearyl benzoate, hexyldecyl benzoate, benzyl benzoate, and the like.

Additional suitable esters include, without limitation, polyglyceryl diisostearate/IPDI copolymer, triisostearoyl polyglyceryl-3 dimer dilinoleate, polyglycerol esters of fatty acids, and lanolin, to name but a few.

Other suitable oils include, without limitation, castor oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, avocado oil, camellia oil, macadamia nut oil, turtle oil, mink oil, soybean oil, grape seed oil, sesame oil, maize oil, rapeseed oil, sunflower oil, cottonseed oil, jojoba oil, peanut oil, olive oil, and combinations thereof.

In one embodiment, the composition may comprise polydecene and/or gylceryl rosinate.

In another embodiment, the composition may comprise an oil selected from the group consisting of octyldodecanol, isododecanol, polyisobutene, polydecene, polyvinylpyrrolidone, mineral oil, and silicone oil.

The oil may be present in the composition, individually or in the aggregate, in an amount sufficient to form a gel with the polyamide gellant. The oil may be present individually or in the aggregate, in an amount between about 0.01% and about 25%, between about 0.1% and about 15%, between about 0.5% and about 10%, or between about 1% and about 8%, or between about 2% about 5% by weight of the composition.

The composition also typically comprises one or more associative rheological modifiers/thickeners. Some suitable associative rheological modifiers/thickeners include, for example, ethoxylated polyurethanes, and cross-linked acrylate copolymers, and acrylic acid homopolymers, as well as any suitable nonionic, cationic, anionic or amphoteric thickeners.

In one embodiment, the associative thickener comprises an alkali-swellable acrylic emulsion polymer, such as Acrylates Copolymer (INCI).

In some embodiments, the associative thickener may comprise one or more of DAITOSOL 5000SJ (INCI: Acrylates/Ethylhexyl Acrylate Copolymer); DAITOSOL 5000AD (INCI: Acrylates Copolymer); SYNTRAN PC 5100 (INCI: Polyacrylate-21 and Acrylates/Dimethylaminoethyl Methacrylate Copolymer); SYNTRAN PC 5208 (INCI: Polyacrylate-15); SYNTRAN PC 5330 (INCI: Polyquaternium-91 and Polyacrylate-15); and SYNTRAN PC 5775 (INCI: Acrylates Ethylhexyl Acrylate/HEMA Copolymer and Acrylates/Diethylamino Methacrylate/Ethylhexyl Acrylate Copolymer).

In other embodiments, the associative thickener may comprise one or more of the following cross-linked acrylate copolymers: CARBOPOL 940, CARBOPOL 941, CARBOPOL AQUA SF-2, and CARBOPOL Aqua SF-1.

Other suitable associative thickeners can include, for example, acrylates/C₁₀₋₃₀ alkyl acrylate crosspolymer (available under the trade names CARBOPOL 1342 and 1382 by Lubrizol Corp.; and PEMULINS TR-1 and TR-2 from BF Goodrich), Acrylates/Steareth-20 Itaconate copolymer (available under the trade name STRUCTURE 2001 from National Starch), Acrylates/Ceteth-20 Itaconate copolymer (available under the trade name STRUCTURE 3001 from National Starch), bentonite, PVM/MA Decadiene Crosspolymer, which is a crosspolymer of methylvinylether/maleic anhydride copolymer cross-linked with 1, 9 decadiene (available under the trade name STABILIZE QM from International Specialties Products), Acrylates/steareth-20 methacrylate copolymer (sold under the trade name ACRYSOL ICS-1 by Rohm and Haas Co.), acrylamide/sodium acrylate copolymer (sold under the trade name HOSTACERIN PN 73 by Hoecsht AG), acrylate copolymer (sold under the trade name ANTIL 208 by Goldschmidt), acrylic acid/acrylonitrogens copolymer (sold under the trade names HYPAN SA-100H, SR-150H supplied by Lipo), Acrylic/acrylate copolymer (sold under the trade names CARBOSET 5, 514, 515, 525, XL-19, XL-19, X1-28, XL-40, 526 by BF Goodrich), Ammonium acrylates/acrylonitrogens copolymer (sold under the trade name HYPAN SS-201 by Lipo), Quaternium-18 Bentonite, which is a sodium salt of crosslinked poly(acrylic acid) (sold under the tradenames PNC 430, PNC 410, PNC 400 by 3V), Stearalkonium Bentonite (sold under the trade name CLAYTON by Southern Clay Products), Quaternium-18 Hectorite (Bentone 38), Stearalkonium Hectorite (Bentone 27), Poly(acrylic acid) (sold under the trade names CARBOPOL 400 by BF and Aquatreat by Alco), trihydroxystearin (commercially available under the trade names THIXICIN by Rheox and Flowtone Southern Clay Products), Dimethylaminoethyl methacrylamide and acrylamide copolymer (SALCARE SC63 from Ciba Specialties), Acrylic polymer anionic or cationic thickening agents (sold under the trade name SYNTHALEN by 3V), Polyacrylate-1 crosspolymer (INCI) (sold under the trade name CARBOPOL Aqua CC by Lubrizol Corp.), Sodium Acrylate copolymer (sold under the trade name TINOVIS ADM by Ciba), and Polyacrylamidomethylpropane Sulfonic Acid (sold under the trade name COSMEDIA HSP-1180 by Cognis Care Chemicals).

Suitable cationic polymer thickeners include, but are not limited to, cationized cellulose, cationized guar gum, diallyly quaternary ammonium salt/acrylamide copolymers, polyquaternium-37 (INCI), and mixtures thereof.

The associative rheological modifier/thickener may be present, individually or collectively, in an amount between about 0.1% and about 20%, or between about 0.5% and about 15%, or between about 1% and about 10%, or between about 3% and about 6% by weight of the composition.

The composition also typically comprises at least one film-forming agent which may improve the wear of the composition, and can confer transfer-resistance to a make-up product. The film-forming agent may be any which is cosmetically acceptable for use around the eye, for example. Examples include polymers such as polyurethane polymers, polyethylene polymers, PVP, copolymers of PVP, ethylene vinyl acetate, dimethicone gum, C₁-C₆ alkyl (meth)acrylate polymer, polyacrylates, polymethacrylates, cellulose polymers, and resins such as trimethylsiloxysilicate.

In one embodiment, the composition comprises a polyurethane film former, for example, those that are formed by reacting a di- or polyisocyanate with a diol and/or polyol), including for example, aqueous polyurethane dispersions. In one embodiment, the film former may comprise a copolymer of adipic acid, dicyclohexylmethane diisocyanate, ethylenediamine, Hexandiol, Neopentyl Glycol and sodium N-(2-aminoethyl)-3-aminoethanesulfonate monomers (INCI: Polyurethane-35; sold by Covestro as BAYCUSAN C1004). In another embodiment, the film former may comprise a copolymer of Hexanediol, Neopentyl Glycol, and Adipic Acid is reacted with hexamethylene diisocyanate, which may be further reacted with N-(2-aminoethyl)-3-aminoethanesulfonic acid and ethylenediamine (INCI: Polyurethane-34; sold by Covestro as BAYCUSAN C1000, and BAYCUSAN C1001). In another embodiment, the film former may comprise a copolymer of 1,4-Butanediol, ethylenediamine, hexamethylene diisocyanate, isophorone diisocyanate, and sodium N-(2-aminoethyl)-3-aminoethane sulfonate monomers (INCI: Polyurethane 32; sold by Covestro as BAYCUSAN C1003). In another embodiment, the film former may comprise waterborne polyurethane dispersion based on adipic acid, 1-6 hexandiol, neopentyl glycol, isophorone diisocyanate, isophorone diamine, N-(2-aminoethyl)-3-aminoethanesulphonicacid, sodium salt (INCI: Polyurethane 48; sold by Covestro as BAYCUSAN C1008).

In other embodiments, the film former may comprise AQUACOAT Gel (INCI: Polyurethane/PEG-6/PEG-90M), and/or ASCENA RC 880 (INCI: Polyurethane/PEG/TMHDI/Hexyldecanol/Octyldodecanol/PG/Aa).

Additional suitable polymeric film formers include, without limitation, acrylic polymers or co-polymers, (meth)acrylates, alkyl (meth)acrylates, polyolefins, polyvinyls, polacrylates, polyurethanes, silicones, polyamides, polyethers, polyesters, fluoropolymers, polyethers, polyacetates, polycarbonates, polyamides, polyimides, rubbers, epoxies, formaldehyde resins, organosiloxanes, dimethicones, amodimethicones, dimethiconols, methicones, silicone acrylates, polyurethane silicones copolymers, cellulosics, polysaccharides, polyquaterniums, and the like. Suitable film formers include those listed in the Cosmetic Ingredient Dictionary and Handbook, 12th Edition (2008), the disclosure of which is hereby incorporated by reference.

Suitable silicone acrylate copolymers include those comprising a poly(alkyl)acrylate backbone and a dimethicone polymer grafted to an alkyl ester side chain, such as the commercially available film former Cyclopentasiloxane (and) Acrylates/Dimethicone Copolymer (KP-545, Shin-Etsu Chemical Co., Ltd) and Methyl Trimethicone (and) Acrylates/dimethicone Copolymer (KP-549, Shin-Etsu Chemical Co., Ltd.).

Other suitable polymeric film formers include, without limitation, Amino Bispropyl Dimethicone, Aminopropyl Dimethicone, Amodimethicone, Amodimethicone Hydroxystearate, Behenoxy Dimethicone, C30-45 Alkyl Dimethicone, C24-28 Alkyl Dimethicone, C30-45 Alkyl Methicone, Cetearyl Methicone, Cetyl Dimethicone, Dimethicone, Dimethoxysilyl Ethylenediaminopropyl Dimethicone, Hexyl Methicone, Hydroxypropyldimethicone, Stearamidopropyl Dimethicone, Stearoxy Dimethicone, Stearyl Methicone, Stearyl Dimethicone and Vinyl Dimethicone. Particularly preferred are silicone polymers, including Methicone (as described by CTFA Monograph No. 1581, which is incorporated herein by reference), Dimethicones (as described by CTFA Monograph No. 840, which is incorporated herein by reference) and Amodimethicones as described by CTFA Monograph No. 189, which is incorporated herein by reference). In some embodiments, the film former comprises a hydrophilic film forming polymer, such as hydroxyethylcellulose or other cellulosics, PVP, and polyvinyl alcohol.

The film former may be present, individually or in the aggregate, in an amount of from about 0.01% to about 50%, or from about 0.05% to about 30%, or from about 0.1% to about 20%, or from about 1% to about 10%, or from about 6% to about 8% by weight of the composition. In some embodiments, the composition may comprise about 1%, or about 2%, or about 3%, or about 4%, or about 5%, or about 6%, or about 7%, or about 8% or about 9%, or about 10%, relative to the total weight of the composition.

In some embodiments, a polyurethane film former, such as polyurethane-35, is present in an amount from about 1% to about 30%, or between about 5% to about 10%, or between about 6% and about 8% by weight of the composition. The specified amount of polyurethane film former is on a solids basis. However, in some embodiments, the polyurethane may be an aqueous dispersion, e.g., a 30-50% (w/w) aqueous dispersion.

The compositions may also comprise any suitable one or more polar solvents, which may be included individually or in the aggregate, in an amount between about 0.1% to about 50%, between about 1% and about 40%, between about 5% and about 30%, or between about 10% and about 20% by weight of the composition.

Particulates may optionally be included. Particulates may include any suitable pigments, lakes, fillers etc. Particulates will typically have a particle size between about 1 nm and about 1 mm. In some embodiments, at least 90% of the volume of particulates has a size (average diameter) greater than about 10 nm. In some embodiments, less than 10% of the total volume of particulates has a size greater than 100 microns. In one embodiment, particulates in the composition comprise those that are spherical and less than about 10 microns in diameter.

In one embodiment, the composition comprises particulates selected from the group consisting of carbon black, glass beads (e.g., borosilicate), iron oxide, silica, talc, and combinations thereof.

For purposes of the current invention, “pigments” shall be defined as organic pigments, inorganic pigments, lakes, pearlescent pigments, and or combinations thereof. Typically the compositions will include pigments to impart a desired color or effect. Color cosmetics, including mascaras, of the current invention may include black including various shades as well as additional known colors for mascaras. In certain, embodiments, the color white may be excluded from the colors of mascara available.

Examples of pigments are inorganic pigments, organic pigments, and/or lakes. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (α-Fe₂O₃, γ-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides as well as composite oxides and composite hydroxides such as iron titanate, cobalt titanate and cobalt aluminate. Non-metal oxides also contemplated to be suitable are alumina and silica, ultramarine blue (i.e., sodium aluminum silicate containing sulfur), Prussian blue, manganese violet, bismuth oxychloride, talc, mica, sericite, magnesium carbonate, calcium carbonate, magnesium silicate, aluminum magnesium silicate, silica, titanated mica, iron oxide titanated mica, bismuth oxychloride, and the like. Organic pigments can include, but are not limited to, at least one of carbon black, carmine, phthalocyanine blue and green pigment, diarylide yellow and orange pigments, and azo-type red and yellow pigments such as toluidine red, litho red, naphthol red and brown pigments, and combinations thereof.

Lakes generally refer to a colorant prepared from a water-soluble organic dye, (e.g., D&C or FD&C) which has been precipitated onto an insoluble reactive or absorptive substratum or diluent. The term “D&C” as used herein means drug and cosmetic colorants that are approved for use in drugs and cosmetics by the FDA. The term “FD&C” as used herein means food, drug, and cosmetic colorants which are approved for use in foods, drugs, and cosmetics by the FDA. Certified D&C and FD&C colorants suitable for precipitation onto the insoluble reactive or absorptive stratum of lakes are listed in 21 C.F.R. §74.101 et seq. and include the FD&C colors Blue 1, Blue 2, Green 3, Orange B, Citrus Red 2, Red 3, Red 4, Red 40, Yellow 5, Yellow 6, Blue 1, Blue 2, Orange B, Citrus Red 2, and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable lakes include, without limitation, those of red dyes from the monoazo, disazo, fluoran, xanthene, or indigoid families, such as Red 4, 6, 7, 17, 21, 22, 27, 28, 30, 31, 33, 34, 36, and Red 40; lakes of yellow pyrazole, monoazo, fluoran, xanthene, quinoline, dyes or salt thereof, such as Yellow 5, 6, 7, 8, 10, and 11; lakes of violet dyes including those from the anthroquinone family, such as Violet 2, as well as lakes of orange dyes, including Orange 4, 5, 10, 11, and the like. Suitable lakes of D&C and FD&C dyes are defined in 21 C.F.R. §82.51.

In addition to the foregoing, the compositions according to the invention may comprise additional pigments, and/or pearlescents. Inorganic pigments include without limitation titanium dioxide, zinc oxide, iron oxides, chromium oxide, ferric blue, mica, bismuth oxychloride, and titinated mica; organic pigments include barium, strontium, calcium or aluminum lakes, ultramarines, and carbon black. In certain, embodiments mascaras of the current invention exclude white pigments (e.g., titanium dioxide, zinc oxide, or calcium carbonate).

Suitable pearling pigments include without limitation bismuth oxychloride, guanine and titanium composite materials containing, as a titanium component, titanium dioxide, titanium lower oxides or titanium oxynitride, as disclosed in U.S. Pat. No. 5,340,569, the contents of which are hereby incorporated by reference. Other suitable pearlescent materials typically are pigments or layers of titanium dioxide on a substrate such as mica, polyethylene terephthalate, bismuth oxychloride, aluminum oxide, calcium borosilicate, synthetic flourophlogopite (synthetic mica), silica, acrylates copolymer, methyl methacrylate, and the like. Interference or pearl pigments may also be included. These are typically comprised of micas layered with about 50 to 300 nm films of TiO₂, Fe₂O₃, or Cr₂O₃ or the like. These include white nacreous materials, such as mica covered with titanium oxide or covered with bismuth oxychloride; and colored nacreous materials, such as titanium mica with iron oxides, titanium mica with ferric blue or chromium oxide, titanium mica with an organic pigment of the aforementioned type.

The pearlescent pigments can be chosen from white pearlescent pigments, such as mica covered with titanium or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

Commercially available pearlescent pigments suitable for the current invention include, but are not limited to, MICAMIRA (Sandream Enterprises), SYNMIRA (Sandream Enterprises), GlassMira (Sandream Enterprises), XIRONA (EMD Performance Chemicals), TIMIRON (EMD Performance Chemicals), COLORONA (EMD Performance Chemicals), RONASTAR (EMD Performance Chemicals), RONAFLAIR (EMD Performance Chemicals), REFLECKS (BASF), DUOCROME (BASF), and CHIONE (BASF).

The pigments may be optionally surface treated to, for example, make the particles more hydrophobic or more dispersible in a vehicle. The surface of the particles may, for example, be covalently or ionically bound to an organic molecule or silicon-based molecule or may be absorbed thereto, or the particle may be physically coated with a layer of material. The surface treatment compound may be attached to the particle through any suitable coupling agent, linker group, or functional group (e.g., silane, ester, ether, etc). The compound may comprise a hydrophobic portion which may be selected from, for example, alkyl, aryl, allyl, vinyl, alkyl-aryl, aryl-alkyl, organosilicone, di-organosilicone, dimethicones, methicones, polyurethanes, silicone-polyurethanes, and fluoro- or perfluoro-derivatives thereof. Other hydrophobic modifiers include, but are not limited, lauroyl lysine, Isopropyl Titanium Triisostearate (ITT), ITT and Dimethicone (ITT/Dimethicone) cross-polymers, ITT and Amino Acid, ITT/Triethoxycaprylylsilane Crosspolymer, waxes (e.g., carnauba), fatty acids (e.g., stearates), HDI/Trimethylol Hexylactone Crosspolymer, PEG-8 Methyl Ether, Triethoxysilane, aloe, jojoba ester, lecithin, perfluoroalcohol phosphate, and Magnesium Myristate (MM). In other embodiments, the pigments or particulates may be surface treated with galactoarabinase or glyceryl rosinate. The pigments and particulates may be surface modified with, for example, fluoropolymers, to adjust one or more characteristics of the colorant as described in, for example, U.S. Pat. Nos. 6,471,950, 5,482,547, and 4,832,944, the contents of which are hereby incorporated by reference. In another embodiment, the pigments or particulates may be surface treated with Disodium Stearoyl Glutamate (and) Aluminum Dimyristate (and) Triethoxycaprylysilane. In one embodiment, the composition comprises a metal oxide surface treated with triethoxycaprylyl silane or trimethoxycaprylyl silane, including in amounts from about 0.1-10% by weight of the treated particulate (e.g., from about 1-5% by weight).

In some embodiments, the pigments include Iron Oxides, Black Oxide of Iron, Brown Iron Oxide, Iron Oxide Red 10-34-PC-2045, Pigment Black 11, Pigment Brown 6, Pigment Brown 7, Pigment Red 101, Pigment Red 102, Pigment Yellow 42, Pigment Yellow 43, Red Iron Oxide, Synthetic Iron Oxide, Yellow Iron Oxide, or carbon black. In some embodiments where carbon black is used as a pigment all or a portion thereof may be dispersed in a suitable synthetic wax.

The amount of all such pigments, individually or in the aggregate, is not particularly restricted. Typically, the particulates, including pigments and/or colorants, may comprise from about 0.01% to about 40% of the composition, from about 0.1% to about 20% by weight of the composition, or from about 1% to about 10% by weight of the composition. In certain embodiments, the composition will contain about 1%, 2.5%, 5%, 7.5%, 10%, 12.5%, or about 15% by weight pigments or other particulates. In embodiments incorporating carbon black as a pigment, the amount of carbon black incorporated may be about 0.005% to about 5%, or about 0.025% to about 1%, or about 1% to about 3% by weight of the composition. When the cosmetic composition is an emulsion, the pigments are added to the phase in which they are most compatible. For example pigments that have a hydrophobic treatment would be incorporated into the lipophilic phase, while pigments having a hydrophilic treatment would be in the aqueous phase. Pigments with silicone coatings would be incorporated into the silicone phase of a silicone-water emulsion.

In some embodiments, the total amount of particulates present in the composition is less than about 25% by weight, or less than about 20% by weight, or less than about 15% by weight, or less than about 12.5% by weight, or less than about 10% by weight, or less than 9% by weight, or less than about 8% by weight, or less than about 7% by weight, or less than about 6% by weight, or less than about 5% by weight, or less than about 4% by weight, or less than about 3% by weight, or less than about 2% by weight, or less than about 1% by weight, or less than about 0.5% by weight of the composition. In some embodiments, the compositions are free of particulates.

In some embodiments, the compositions may comprise other ingredients that contribute structure to the composition, including but not limited to clays, gums, fibers, and powders, which may be present, individually or in the aggregate, in an amount between about 0.01% to about 25% by weight of the composition.

The compositions may, for example, comprise an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. The emulsion may include an emulsifier, such as a nonionic, anionic or amphoteric surfactant, for example in an amount sufficient to stabilize the emulsion (e.g., 0.001-10% by weight).

The compounds suitable for use in the oil phase include any of the oils described herein. The oil-containing phase may be composed of a singular oil or mixtures of different oils. The oil phase may comprise from about 1-99% (or about 5-95%, or about 10-90%, or about 20-80%) by weight of the emulsion. The aqueous phase may comprise from about 1-99% or about 5-95%, or about 10-90%, or about 20-80% by weight of the emulsion.

The aqueous phase of the emulsion in one embodiment may have one or more organic compounds, including humectants (such as butylene glycol, propylene glycol, Methyl gluceth-20, and glycerin); other water-dispersible or water-soluble components including thickeners such as veegum or hydroxyalkyl cellulose; gelling agents, such as high MW polyacrylic acid, i.e. CARBOPOL 934; and mixtures thereof. In one embodiment, the aqueous phase may include a film forming polymer, for example an acrylate copolymer. In one embodiment, an acrylates copolymer is characterized as having a viscosity of about 25 cps in a 30% aqueous solution. The emulsion may have one or more emulsifiers capable of emulsifying the various components present in the composition.

Non-limiting emulsifiers include emulsifying waxes, emulsifying polyhydric alcohols, polyether polyols, polyethers, mono- or di-ester of polyols, ethylene glycol mono-stearates, glycerin mono-stearates, glycerin di-stearates, silicone-containing emulsifiers, soya sterols, fatty alcohols such as cetyl alcohol, acrylates, fatty acids such as stearic acid, fatty acid salts, and mixtures thereof. Emulsifiers may include soya sterol, cetyl alcohol, stearic acid, emulsifying wax, acrylates, silicone containing emulsifiers and mixtures thereof. Other specific emulsifiers that can be used in the composition of the present invention include, but are not limited to, one or more of the following: C₁₀₋₃₀ alkyl acrylate crosspolymer; Dimethicone PEG-7 isostearate, acrylamide copolymer; mineral oil; sorbitan esters; polyglyceryl-3-diisostearate; sorbitan monostearate, sorbitan tristearate, sorbitan sesquioleate, sorbitan monooleate; glycerol esters such as glycerol monostearate and glycerol monooleate; polyoxyethylene phenols such as polyoxyethylene octyl phenol and polyoxyethylene nonyl phenol; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2, Steareth-10, and Steareth-20, to name a few. Additional emulsifiers are provided in the NCI Ingredient Dictionary and Handbook 11^{th} Edition 2006, the disclosure of which is hereby incorporated by reference in its entirety.

Water-in-silicone emulsions may be emulsified with a nonionic surfactant (emulsifier) such as, for example, polydiorganosiloxane-polyoxyalkylene block copolymers, including those described in U.S. Pat. No. 4,122,029, the disclosure of which is hereby incorporated by reference in its entirety. These emulsifiers generally comprise a polydiorganosiloxane backbone, typically polydimethylsiloxane, having side chains comprising -(EO)ₘ— and/or —(PO)ₙ— groups, where EO is ethyleneoxy and PO is 1,2-propyleneoxy, the side chains being typically capped or terminated with hydrogen or lower alkyl groups (e.g., C₁₋₆, typically C₁₋₃). Other suitable water-in-silicone emulsifiers are disclosed in U.S. Pat. No. 6,685,952, the disclosure of which is hereby incorporated by reference herein. Commercially available water-in-silicone emulsifiers include those available from Dow Corning under the trade designations 3225C and 5225C FORMULATION AID; SILICONE SF-1528 available from General Electric; ABIL EM 90 and EM 97, available from Goldschmidt Chemical Corporation (Hopewell, Va.); and the SILWET series of emulsifiers sold by OSI Specialties (Danbury, Conn.).

Examples of water-in-silicone emulsifiers include, but are not limited to, dimethicone PEG 10/15 crosspolymer, dimethicone copolyol, cetyl dimethicone copolyol, PEG-15 lauryl dimethicone crosspolymer, laurylmethicone crosspolymer, cyclomethicone and dimethicone copolyol, dimethicone copolyol (and) caprylic/capric triglycerides, polyglyceryl-4 isostearate (and) cetyl dimethicone copolyol (and) hexyl laurate, and dimethicone copolyol (and) cyclopentasiloxane. In one embodiment examples of water-in-silicone emulsifiers include, without limitation, PEG/PPG-18/18 dimethicone (trade name 5225C, Dow Corning), PEG/PPG-19/19 dimethicone (trade name BY25-337, Dow Corning), Cetyl PEG/PPG-10/1 dimethicone (trade name ABIL EM-90, Goldschmidt Chemical Corporation), PEG-12 dimethicone (trade name SF 1288, General Electric), lauryl PEG/PPG-18/18 methicone (trade name 5200 FORMULATION AID, Dow Corning), PEG-12 dimethicone crosspolymer (trade name 9010 and 9011 silicone elastomer blend, Dow Corning), PEG-10 dimethicone crosspolymer (trade name KSG-20, Shin-Etsu), dimethicone PEG-10/15 crosspolymer (trade name KSG-210, Shin-Etsu), and dimethicone PEG-7 isostearate.

The emulsifiers typically will be present in the composition in an amount effective to disperse the discontinuous phase into the continuous phase, typically from about 0.001% to about 10% by weight, in another embodiment in an amount from about 0.01% to about 5% by weight, and in a further embodiment in an amount below 1% by weight.

The aqueous phase of the emulsion may include one or more volatile solvents, including lower alcohols, such as ethanol, isopropanol, and the like. The volatile solvent may also be a cosmetically acceptable ester such as butyl acetate or ethyl acetate; ketones such as acetone or ethyl methyl ketone; or the like. The volatile solvents are generally present in an amount of 25% or less by weight of the composition. In other embodiments the volatile solvent is present in an amount of less than 15%, less than 10%, or less than 5% by weight of the composition. In another embodiment the compositions do not contain a volatile solvent.

The non-aqueous phase will typically comprise from about 10% to about 90%, about 30% to about 80%, or from about 50% to about 70% by weight, based on the total weight of the emulsion, and the aqueous phase will typically comprise from about 10% to about 90%, about 30% to about 80%, or from about 40% to about 70% by weight of the total emulsion. In one embodiment of the invention the mascara composition is a water-in-silicone emulsion in which the aqueous phase is from about 20% to about 60% by weight of the total composition and the non-aqueous silicone phase is from about 40% to 80% by weight of the total composition. In one embodiment of the invention the mascara composition is a water-in-oil or oil-in-water emulsion in which the aqueous phase is about 60% by weight of the total composition and the non-aqueous oil phase is about 40% by weight of the total composition.

The compositions may, for example, be anhydrous or may be substantially anhydrous. “Substantially anhydrous” as used herein means containing less than 5% by weight water. In other embodiments, the compositions will comprise less than about 2.5% by weight water, or less than 2% by weight water, or less than about 1% by weight water, or less than 0.25% by weight water. In some embodiments, the compositions may be anhydrous. The term “anhydrous” as used herein means that no water is added to the composition and that only that amount of moisture absorbed from the atmosphere will be present in the composition.

An anhydrous vehicle may include without limitation, vegetable oils; esters including emollient esters, such as octyl palmitate, isopropyl myristate and isopropyl palmitate; ethers such as dicapryl ether; fatty alcohols such as cetyl alcohol, stearyl alcohol octyldodecanol and behenyl alcohol; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; and the like. Suitable hydrophobic hydrocarbon oils may be saturated or unsaturated, have an aliphatic character and be straight or branched chained or contain alicyclic or aromatic rings. Such components may be present individually or in the aggregate, in an amount between about 0.01% to about 20% by weight of the composition.

Mascara compositions of the current invention may have a consistency of a liquid and/or viscous liquid. The hardness of the mascara may be measured by penetrating a probe into the composition. In particular, a texture analyzer (for example TA-XT2i from Rheo) equipped with a 2 mm needle probe may be used. The texture analyzer may be set to: Measurement Mode: Force in Compression; Test Speed: 1.0 mm/s; Distance: 5 mm; and Trigger Force: 5 g. The mascara compositions of the current invention may have a penetrating force of less than about 15 g and in other embodiments the penetrating force may be less than about 10 g. The hardness value may be between about 1 g and 15 g.

Additionally, the compositions of the current invention may exhibit a viscosity between about 250,000 centipoise and about 2,000,000 centipoise, in another embodiment between about 500,000 centipoise and about 1,750,000 centipoise; and about 750,000 centipoise and about 1,500,000 centipoise. The viscosity of the composition may be determined by using a Brookfield DV-E viscometer rotating at 4 rpms with a T-bar E spindle, at 25° C. In one embodiment, the composition is in the form of a liquid having a viscosity between about 250,000 cps and about 2,000,000 cps.

In an additional embodiment of the invention, an additional polyamide resin may provide additional structural integrity to the polymeric gellant. Polyamide resins are high molecular weight polymers which feature amide linkages along the molecular chain. These polymers contain monomers of amides joined by peptide bonds. They can occur both naturally and artificially. Such polymers are made through step growth polymerization or solid phase synthesis. In some cases, examples of polyamide resins are nylons and aramids. Due to their extreme durability and strength, polyamide resins are typically utilized in textiles, plastics and various automotive applications. In the composition of the present invention the polyamide resin also provides a degree of gloss or shine to the composition and adhesion to the target substrate.

One skilled in the art will be able to select suitable additional polyamide resins, and many suitable polymers are disclosed in the CTFA Handbook, 12'h Ed. 2008, the disclosure of which is hereby incorporated by reference. These include, without limitation, Ethylenediamine/Dimer Tallate Copolymer Bis-Hydrogenated Tallow Amide, Ethylenediamine/Stearyl Dimer Dilinoleate Copolymer, Ethylenediamine/Stearyl Dimer Tallate Copolymer, etc.

An additional polyamide resin, or a combination of compatible polyamide resins, may be present in an amount ranging from about 0.01% to about 25% by weight, about 1% to about 20% by weight, or about 5% to about 15% of the composition.

The compositions of the invention may also comprise additional thickeners or viscosifying agents, such as, for example, a polysaccharide or non-polysaccharide thickener. The composition may also comprise silica, xanthan gum, CMC, acrylic acid polymers, bentone, triglycerides, aluminum stearate, C₁₈-C₃₆ acid glycol esters, glyceryl tribehenate, glycerol monostearate, alginates, carbomers, celluloses, hydrated magnesium and aluminium silicates, or calcium silicates, or the like. Oil-soluble rheology modifiers such as trihydroxystearin and/or 12-hydroxystearic acid may also be included. When present, additional thickeners may comprise, individually or in the aggregate, from about 0.01% to about 15% by weight of the composition, more typically from about 1% to about 5% by weight of the composition.

Compounds commonly used in the cosmetic arts for preventing or reducing fungal, bacterial, or microorganismal growth are also added to the composition of the disclosure. By including these compounds, the shelf life of the composition is lengthened. These anti-fungal and anti-microorganisms include but are not limited to methyl paraben, butyl paraben, sodium dehydroacetate, etc. The amounts of these ingredients that may be used within the inventive composition effectively reduce fungal, bacterial, and/or microorganismal growth without negatively affecting the components of the inventive composition or its desired effects.

The compositions of the invention may optionally comprise other active and inactive ingredients typically associated with the intended cosmetic or personal care products. Suitable other ingredients include, but are not limited to, amino acids, antioxidants, conditioners, chelating agents (e.g., sodium hexametaphosphate), colorants, emollients, emulsifiers, excipients, fillers, fragrances, gelling agents, humectants, pH adjusters (e.g., triethanolamine), minerals, moisturizers, photostabilizing agents (e.g., UV absorbers), sunscreens, preservatives, stabilizers, staining agents, surfactants, viscosity and/or rheology modifiers, vitamins, waxes and mixtures thereof. In one embodiment, the compositions may comprise an anti-foaming agent, for example, Dimethicone/Silica/Sorb. Stearate/PEG 40 Stearate blend. The additional components may be present individually or in the aggregate, in an amount between about 0.0001% and about 25%, between about 0.01% and about 15%, between about 0.1% and about 10%, or between about 1% and about 5% by weight of the composition.

All ingredients useful herein may be categorized or described by their postulated mode of action. However, it is to be understood that the ingredients can, in some instances, provide more than one cosmetic and/or therapeutic benefit or operate via more than one mode of action. Therefore, classifications herein are made for the sake of convenience and are not intended to limit an ingredient to the particularly stated application or applications listed.

It should be noted that although reference may be made throughout to mascara compositions, the inventive compositions and methods are applicable to any kind of cosmetic composition, including, for example, lipstick, lip color, lip gloss, nail polish, foundation, eye liner, and the like, as well as to any suitable personal care product, such as day creams or lotions, night creams or lotions, sunscreen lotions, sunscreen creams, sunscreen sprays or oils and other SPF products, moisturizers, salves, ointments, gels, body milks, artificial tanning compositions, facial masks, depilatories, shampoos, conditioners, hair masks, and the like.

Any of the compositions and ingredients therefor disclosed in U.S. Provisional Patent Application Ser. Nos. 61/789,975 and 61/790,104, both filed on Mar. 15, 2013, and in PCT/US14/27609 and PCT/US14/27692, both filed on Mar. 14, 2014, the entire contents of which are incorporated by reference herein for all purposes, are contemplated to be suitable for practice of the present invention.

The composition of the invention should be cosmetically or dermatologically acceptable, i.e., it should contain a non-toxic physiologically acceptable medium and should be able to be applied to the eyelashes of human beings. For the purposes of the invention, the expression “cosmetically acceptable” means a composition of pleasant appearance, odor, feel and taste.

Methods for imparting the appearance of both volume and length to keratin fibers are also provided. Any keratin fibers may be used as a substrate, such as, for example, eyelashes, eyebrows, or hair on the head (scalp). The methods generally comprise applying to the surfaces of a keratin fiber a composition of the invention on at least a portion of surfaces of the fibers (e.g., along at least a portion, or along a substantial portion, or along a majority of, or along substantially the entire length and/or circumference of the fiber) and drawing an amount of the composition beyond the terminal ends of the fibers. A portion of the composition is extended out beyond the tip of the keratin fiber, for example between about 0.1 mm to about 1 cm or more, beyond the tip of the keratin fiber and allowed to harden into a solid extension thereof. In other embodiments, the tip of the keratin fiber is extended between about 0.1 and 5 mm, or between about 0.5 and 1 mm after a dry film forms on the treated fiber.

The compositions of the invention may be characterized as having superior extensional viscosity compared with conventional mascara formulations, for example, and may exhibit a first normal stress difference (as defined in Example 2) of at least 35 Pa, or at least 40 Pa, or at least 45 Pa, or at least 50 Pa, or at least 55 Pa, or at least 60 Pa.

The compositions may be applied to keratin fibers, for example with a brush, comb, or other suitable applicator (including, for example, any known mascara or cosmetics applicator). The compositions are applied to form a film or coating on the surface of individual fibers (e.g., lashes) along part or all of the length of the fiber. After the compositions have cured or partially cured, for example, by partial or complete evaporation of solvents and other volatiles, a dry film forms on the treated keratin fibers. This film is flexible and substantially resistant to flaking and/or breakage following moving or stretching of the treated lashes.

Flexibility of compositions of the invention may be characterized based on a flexibility test such as that described in Example 3. In one embodiment, a mascara film of the invention is characterized as losing less than less than 20%, or less than 15%, or less than 10%, or less than 5%, or less than 4%, or less than 3% or less than 2%, or less than 1% of its original weight after forming a dry film on a treated keratin fiber, such as an eyelash according to the flexibility test.

In another embodiment, the invention relates to a method for coloring a human integument, including keratin fibers, comprising applying to the human integument a composition of the invention to form a film thereon. A human integument may include skin, lips, nails, hair, and other keratinous surfaces. As used herein, the term “keratinous surface” refers to keratin-containing portions of the human integumentary system, which includes, but is not limited to, skin, lips, hair (including hair of the scalp, eyelashes, eyebrows, facial hair, and body hair such as hair of the arms, legs, etc.), and nails (toenails, fingernails, cuticles, etc.) of mammalians, preferably humans. In a further embodiment, the cosmetic compositions may impart color (e.g., black color) to the human integument (e.g., eyelashes).

A person skilled in the art will take care to select the optional additives and/or the amount thereof such that the advantageous properties of the composition according to the invention are not, or are not substantially, adversely affected by the envisaged addition. It is further understood that the other cosmetic ingredients and adjuvants introduced into the composition must be of a kind and quantity that are not detrimental to the advantageous effect which is sought herein according to the invention.

The following examples describe specific aspects of the compositions of the present invention to illustrate the invention and provide a description for those skilled in the art. The Examples should not be construed as limiting the invention as the examples merely provide specific methodology useful in the understanding and practice of the invention and its various aspects.

### EXAMPLES

### Example 1: Mascara Formulations

Three mascara compositions (A, B, and C) according to the current invention are provided in Table 3.

TABLE 3

| | A | B | C |
|---|---|---|---|
| Ingredient | (weight %) | (weight %) | (weight %) |
| Aqueous Phase | | | |
| Water | q.s. | q.s. | q.s. |
| Film former | 1.0 | 1.0 | 1.0 |
| pH adjuster | 3.0 | 3.0 | 3.0 |
| Preservative | 0.5 | 0.5 | 0.5 |
| Anti-foaming agent | 0.1 | 0.1 | 0.1 |
| Chelating agent | 0.25 | 0.25 | 0.25 |
| Pigments | 6.5 | 6.5 | 6.5 |
| Thickener | 6.0 | 8.0 | 6.0 |
| Polyurethane-35* | 15 | 15 | 20 |
| Oil Phase | | | |
| Emulsifier | 6.0 | 6.0 | 6.0 |
| Oil | 1.5 | 1.5 | 1.5 |
| Bis-Stearyl Ethylenediamine/ | 3.0 | 3.0 | 2.0 |
| Neopentyl Glycol/Stearyl | | | |
| Hydrogenated Dimer Dilinoleate | | | |
| copolymer (Gellant) | | | |
| Film former | 2.0 | 2.0 | 2.0 |
| Beeswax (Soft wax) | 14.0 | 14.0 | 14.0 |
| Bis-PEG-12 dimethicone beeswax | 1.45 | 1.45 | 1.45 |
| (Soft wax) | | | |
| Polyurethane- | 1.0 | 1.0 | 1.0 |
| PEG/TMHDI/Hexyldecanol/ | | | |
| Octyldodecanol blend | | | |
| (Associative thickener) | | | |
| Total | 100 | 100 | 100 |

*40% by weight solids dispersed in water.

### Example 2: First Normal Stress Difference

The first normal stress difference (N₁) for a mascara composition of the invention (mascara composition A in Table 3) and a commercial mascara is assessed as follows. The first normal stress difference is calculated from the formula: N₁=2Jₑ⁰η²γ² where Jₑ⁰=recoverable compliance; η=steady shear creep viscosity; and γ²=steady shear creep shear rate. A higher first normal stress difference indicates a greater extensional viscosity, and therefore a greater ability to lengthen a treated keratin fiber.

The first normal stress difference of the of mascara composition A in Table 3 was 59.6 Pa. In contrast, the first normal stress difference of a commercial mascara was 8.91 Pa. The higher first normal stress difference of the inventive mascara indicates that it possesses higher extensional viscosity than the commercial mascara, and therefore possesses a superior ability to lengthen a treated keratin fiber. In various embodiments, the first normal stress difference will be greater than 20, 30, 40, or 50 Pa.

### Example 3: Flexibility Test

A flexibility test was used to assess the flexibility of a dry film of the invention (mascara composition A in Table 3), and of a commercial mascara. For each test, a strip of latex band was weighed, and 40 mg of mascara was applied to the band, and the weight recorded. The film is left to dry completely overnight at ambient temperature, and the weight is again recorded. The band is then stretched ten times. After ten stretches, any loose product is removed with a small brush. The weight is recorded again, and the percentage of the weight lost by the film after stretching is calculated. This test was run five times for each mascara, and the average of those trials was calculated. A lower average percent weight loss of the film indicates that the dried film experienced less flaking and peeling, and that the dried film therefore has greater flexibility than a film that experienced greater weight loss.

The commercial mascara film lost an average of 88.9% of its original weight, whereas the inventive mascara film lost an average of only 3.2% of its original weight after the flexibility test. These results indicate that the inventive mascara is substantially more flexible and resistant to breakage and flaking compared to the commercial mascara.

The invention described and claimed herein is not to be limited in scope by the specific embodiments herein disclosed since these embodiments are intended as illustrations of several aspects of the invention. Any equivalent embodiments are intended to be within the scope of this invention. Indeed, various modifications of the invention in addition to those shown and described therein will become apparent to those skilled in the art from the foregoing description. Such modifications are also intended to fall within the scope of the appended claims. All publications cited herein are incorporated by reference in their entirety.

1. A composition for application to keratin fibers comprising:
(a) between about 0.1% and about 10% by weight of a polyamide gellant;
(b) between about 0.1% and 10% by weight of an oil capable of forming a gel with said polyamide gellant;
(c) between about 0.1% and 10% by weight of an associative rheological modifier;
(d) between about 0.1% and about 20% by weight of a wax component comprising one or more waxes having a melting point of less than 70° C.; and
(e) between about 0.1% and about 30% by weight of a polyurethane film former.

2. The composition according to claim 1, wherein the composition is characterized by a first normal stress difference of at least 35 Pascals.

3. The composition according to claim 1, wherein said polyamide gellant comprises an ester terminated poly(ester-amide) polymer.

4. The composition according to claim 3, wherein said ester terminated poly(ester-amide) polymer comprises Bis-Stearyl Ethylenediamine/Neopentyl Glycol/Stearyl Hydrogenated Dimer Dilinoleate copolymer (INCI).

5. The composition according to claim 1, wherein said associative rheological modifier comprises a cross-linked acrylates copolymer.

6. The composition according to claim 1, wherein said one or more waxes is selected from the group consisting of beeswax, paraffin wax, bleached beeswax, ozokerite, sorbitol beeswax, silicone wax, and PEG-modified beeswax.

7. The composition according to claim 1, further comprising between about 0.05% and about 10% by weight dibutyl laurolyl glutamide and/or dibutyl ethylhexanoyl glutamide.

8. The composition according to claim 1, further comprising between about 0.1% and about 50% by weight of a polar solvent.

9. The composition according to claim 1, further comprising between about 1% and about 20% by weight iron oxide and/or carbon black.

10. The composition according to claim 1, wherein the composition is a mascara.

11. The composition according to claim 1, further comprising polydecene.

12. The composition according to claim 1, further comprising glyceryl rosinate.

13. The composition according to claim 1, wherein said composition is substantially free of waxes having a melting point of greater than 75° C.

14. The composition according to claim 1, wherein said composition is free of waxes having a melting point of greater than 75° C.

15. A method of imparting length and volume to a keratin fiber, comprising applying to said keratin fiber a composition comprising:
a) between about 0.1% and about 10% by weight of a polyamide gellant;
(b) between about 0.1% and 10% by weight of an oil capable of forming a gel with said polyamide gellant;
(c) between about 0.1% and 10% by weight of an associative rheological modifier;
(d) between about 0.1% and about 20% by weight of a wax component comprising one or more waxes having a melting point of less than 70° C.; and
(e) between about 0.1% and about 30% by weight of a polyurethane film former.

16.-23. (canceled)

24. The method according to claim 15, wherein the composition is characterized by a first normal stress difference of at least 35 Pascals.

25. The composition according to claim 15, wherein said polyamide gellant comprises an ester terminated poly(ester-amide) polymer.

26. The composition according to claim 25, wherein said ester terminated poly(ester-amide) polymer comprises Bis-Stearyl Ethylenediamine/Neopentyl Glycol/Stearyl Hydrogenated Dimer Dilinoleate copolymer (INCI).

27. The composition according to claim 15, wherein said associative rheological modifier comprises a cross-linked acrylates copolymer.

28. The composition according to claim 15, wherein said one or more waxes is selected from the group consisting of beeswax, paraffin wax, bleached beeswax, ozokerite, sorbitol beeswax, silicone wax, and PEG-modified beeswax.

29. The composition according to claim 15, further comprising between about 0.05% and about 10% by weight dibutyl laurolyl glutamide and/or dibutyl ethylhexanoyl glutamide.

30. The composition according to claim 15, further comprising between about 0.1% and about 50% by weight of a polar solvent.

31. The composition according to claim 15, further comprising between about 1% and about 20% by weight iron oxide and/or carbon black.

32. The composition according to claim 15, wherein the composition is a mascara.
